(19) United States
(12) Patent Application Publication
(10) Pub. No.: US 2012/0039823 A1
(43) Pub. Date: Feb. 16, 2012

(54) PREPARATIONS FOR REDUCING ITCHING AND OTHER PARAESTHESIAS WITH (1R,2S,5R)-2-ISOPROPYL-5-METHYL-N-(2-(PYRIDIN-2-YL)ETHYLCYCLOHEXANECARBOXAMIDE AND/OR (1R,2S,5R)-N-(4-(CYANOMETHYL)-PHENYL)-2-ISOPROPYL-5-METHYLCYCLOHEXANECARBOXAMIDE AND/OR (1R,2S,5R)-N-(4-(CYANOMETHYL)-PHENYL)-2ISOPROPYL-5-METHYLCYCLOHEXANECARBOXAMIDE

(75) Inventors: Ludger Kolbe, Dohren (DE); Julia Eckert, Hamburg (DE); Gitta Neufang, Hamburg (DE); Stefanie Knaupmeier (Hoeltkemeier), Hamburg (DE); Nils Peters, Todendorf (DE)

(73) Assignee: BEIERSDORF AG, Hamburg (DE)

(21) Appl. No.: 12/933,231

(22) PCT Filed: Feb. 3, 2009
(86) PCT No.: PCT/EP2009/000687

§ 371 Date: Dec. 13, 2010

(30) Foreign Application Priority Data
Mar. 20, 2008 (DE) 10 2008 015 425.3

(51) Int. Cl. A61K 8/49; A61K 31/277; A61P 17/04; A61Q 17/04; A61Q 19/00; A61P 17/00; A61K 31/4402; A61K 8/40

(52) U.S. Cl. 424/59; 514/357; 514/521

(57) ABSTRACT

Use of topical, cosmetic or dermatological preparations with a content of (1R,2S,5R)-2-isopropyl-5-methyl-N-(2-(pyridin-2-yl)ethylcyclohexanecarboxamide and/or (1R,2S,5R)-N-(4-(cyanomethyl)-phenyl)-2-isopropyl-5-methylcyclohexyl carboxamide for reducing itching.

The present invention relates to cosmetic and dermatological preparations for reducing dermatological paraesthesias.

The skin of older people, which is also referred to as aged skin, differs significantly from young skin. The moisture content and fat content is reduced, the skin dries out and begins to stretch taut and to itch. Itchy skin can also be produced by UV radiation. A slight sunburn likewise manifests itself in dermatological paraesthesias, such as a taut feeling, redness, hypersensitivity and itching.

Itching is an independent sensation of the skin and occurs independently of pain sensation. The surface nerve endings in the skin react to many different neurotransmitters from the skin and the blood with the development of itching. The different sensations of itching such as pure itch, stabbing or burning itch, painful tingling, etc. are explained by the different neurotransmitters. The itching sensation is transported via the spinal nerves to the brain where the need to scratch is directly triggered.

Dry skin, which is caused by a disturbance of the skin's protective function, is very often the reason for itching. Itching occurs particularly intensively with very dry skin, particularly in old age, with neurodermatitis, psoriasis and diabetes.

The itching mechanism runs like a cycle: itching leads almost unavoidably to scratching. The area of skin affected is thereby additionally irritated. Inflammation neurotransmitters are released, which intensify the existing itch still further, and thus can result in a mechanical injury to the skin. An unending cycle begins: itching—scratching—itching . . . .

In order to obtain immediate relief, this cycle must be effectively broken with immediate effect.

It is known from the literature that ethoxylated compounds, such as, e.g., laureth-9 (Fiedler, Lexikon der Hilfsstoffe für Pharmazie und Kosmetik und angrenzende Gebiete [Encyclopedia of Auxiliary Agents for Pharmacy and Cosmetics and Related Fields], Verlag: Editio Cantor Aulendorf, 3rd edition, Vol. 1, p. 540) or polyoxyethylene (20) sorbitan monolaurate (manufacturer's information) or materials from other substance classes, e.g., citric esters (Fiedler, Lexikon der Hilfsstoffe für Pharmazie und Kosmetik und angrenzende Gebiete, Verlag: Edito Cantor Aulendorf, 3^{rd} edition, Vol. 1, p. 309) have a certain itch-reducing effect.

Furthermore, local anesthetics and polidocanol are used.

The use of barrier-strengthening active substances, such as, e.g., omega-6 fatty acids from natural oil of evening primrose, is also advantageous.

Nevertheless, previous itch-relieving cosmetic and/or pharmaceutical compositions were not satisfactory because their effects quickly abate.

There has been no lack of attempts to reduce the itching of itching skin by the application of a preparation. There have also been attempts to find preparations that reduce itching. This was attempted in particular for preparations for the care of aging skin or sun-damaged skin.

One object of the present invention was to remedy the drawbacks of the prior art and to reduce the itching and other problems that can occur with aged skin or due to sunburn by means of preparations that are easy to produce, are tolerated well and are odorless, do not have an irritant effect on the skin or mucous membranes and that provide a pleasant cooling when used according their intended application.

However, it was surprising and not foreseeable for one skilled in the art that the use of topical cosmetic or dermatological preparations with a content of (1R,2S,5R)-2-isopropyl-5-methyl-N-(2-(pyridin-2-yl)ethylcyclohexanecarboxamide and/or (1R,2S,5R)-N-(4-(cyanomethyl)-phenyl)-2-isopropyl-5-methylcyclohexanecarboxamide for reducing itching, for reducing paraesthesias with dry skin and with aged skin and/or for reducing dermatological paraesthesias caused by sunburn eliminate the disadvantages of the prior art.

Surprisingly, (1R,2S,5R)-2-isopropyl-5-methyl-N-(2-(pyridin-2-yl)ethyl-cyclohexanecarboxamide

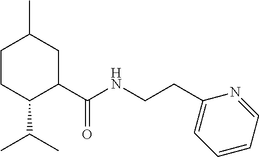

and (1R,2S,5R)-N-(4-(cyanomethyl)-phenyl)-2-isopropyl-5-methylcyclohexane-carboxamide are suitable for the use according to the invention.

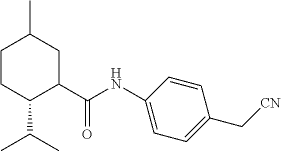

These itch-reducing, cooling substances can be used alone or in combination with other cooling substances.

Examples of known cooling substances are menthol, menthone, ethyl menthanecarboxamido acetate (WS-5), isopulegol (Coolact P), menthanediol (Coolact 38D), N,2,3-trimethyl-2-isopropylbutanamide (WS-23), ethyl menthane carboxamide (WS-3), menthone glycerine acetal (Frescolat MGA) or mono-menthyl succinate (Physcool).

The preparations are easy to formulate and do not make any great demands on production operations.

The production of an O/W emulsion is preferred. The cooling substances are pre-dissolved and added to the emulsion at approx. 40° C.

It is preferred if additionally menthoxypropanediol is contained. Menthoxypropanediol

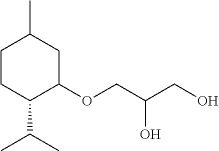

has a long-lasting itch-reducing effectiveness. It is particularly preferred if the preparations contain 0.001-10% by weight, particularly preferably 0.01-1% by weight of (1R,2S,5R)-2-isopropyl-5-methyl-N-(2-(pyridin-2-yl)ethylcyclohexanecarboxamide and/or (1R,2S,5R)-N-(4-(cyanomethyl)-phenyl)-2-isopropyl-5-methylcyclohexane-carboxamide, based on the total weight of the preparations. It is further particularly preferred if the preparations contain 0.001-10% by weight, particularly preferably 0.01-1% by weight of menthoxypropanediol, based on the total weight of the preparations. It is preferred thereby if the preparations contain sunscreen agents. It is furthermore preferred if the preparations contain antioxidants. It is furthermore preferred if the preparations contain vitamins.

For application, the cosmetic and dermatological preparations according to the invention are applied onto the skin and/or the hair in sufficient quantities in the usual manner for cosmetics.

The cosmetic and dermatological preparations according to the invention can comprise cosmetic auxiliaries such as are customarily used in such preparations, e.g., preservatives, bactericides, perfumes, substances to prevent foaming, dyes, pigments that have a coloring action, thickeners, moisturizing substances and/or humectant substances, fats, oils, waxes or other customary constituents of a cosmetic or dermatological formulation, such as alcohols, polyols, polymers, thickeners, sunscreen filters, fillers, foam stabilizers, electrolytes, organic solvents or silicone derivatives.

An additional content of conventional antioxidants is generally preferred, According to the invention all of the antioxidants suitable or customary for cosmetic and/or dermatological applications can be used as favorable antioxidants.

The quantity of the above-referenced antioxidants (one or more compounds) in the preparations is preferably 0.001 to 30% by weight, particularly preferably 0.05-20% by weight, in particular 0.5-10% by weight, based on the total weight of the preparation.

If the cosmetic or dermatological preparation for the purposes of the present invention is a solution or emulsion or dispersion, solvents which can be used are:
water or aqueous solutions;
oils, such as triglycerides of capric acid or of caprylic acid, alkyl benzoates or other esters of fatty acids or plant oils, such as sunflower oil, oil of evening primrose, grapeseed oil, apricot kernel oil, argan oil or almond oil;
fats, waxes and other natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids;
alcohols, diols or polyols of low carbon number, and ethers thereof, preferably ethanol, isopropanol, propylene glycol, glycerol, ethylene glycol, pentanediol, methylpropanediol, ethylene glycol monoethyl or monobutyl ether, diethylene glycol monomethyl or monoethyl ether and analogous products.

In particular, mixtures of the abovementioned solvents are used. In the case of alcoholic solvents, water may be a further constituent.

The oil phase of the preparations preferred according to the invention is advantageously selected from the group of esters of saturated and/or unsaturated, branched and/or unbranched alkane carboxylic acids of a chain length of from 3 to 30 C atoms and saturated and/or unsaturated, branched and/or unbranched alcohols of a chain length of from 3 to 30 C atoms, from the group of esters of aromatic carboxylic acids and saturated and/or unsaturated, branched and/or unbranched alcohols of a chain length of from 3 to 30 C atoms. Such ester oils can then advantageously be chosen from the group of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids of a chain length of from 8 to 24, in particular 12-18, C atoms. The fatty acid triglycerides can, for example, be chosen advantageously from the group of synthetic, semisynthetic and natural oils, e.g. olive oil, sunflower oil, soya oil, peanut oil, rapeseed oil, almond oil, palm oil, coconut oil, palm kernel oil and the like.

Any mixtures of such oil and wax components can also be used advantageously for the purposes of the present invention.

Advantageous preservatives for the purposes of the present invention are, for example, benzyl alcohol, sorbic acid and salts thereof, formaldehyde donors (such as, for example, diazolidinyl urea (trade name Germall II from ISP), imidazolidinyl urea (trade name Germall 115) or DMDM hydantoin, which is available, for example, under the trade name Glydant™ from Lonza), methyl isothiazolinone and corresponding derivatives (trade name Kathon CG), iodopropynyl butylcarbamates (e.g. those available under the trade names Glycacil-L, Glycacil-S from Lonza, and/or Dekaben LMB from Jan Dekker), parabens (i.e., p-hydroxybenzoic acid alkyl esters, such as methyl- ethyl-, propyl- and/or butylparaben), phenoxyethanol, ethanol, triclosan, benzoic acid and the like. In addition, according to the present invention, the preservative system usually also advantageously comprises substances that improve the effectiveness of classic preservatives, such as, for example, hexanediol, pentanediol, butylene glycol and methylpropanediol.

Suitable propellants for cosmetic and/or dermatological formulations according to the invention which can be sprayed from aerosol containers include the customary known readily volatile, liquefied propellants, for example, hydrocarbons (propane, butane, isobutane), which can be employed individually or as mixtures with one another. Compressed air can also advantageously be used.

Of course, one skilled in the art is aware that there are propellant gases that are nontoxic per se and would in principle be suitable for realizing the present invention in the form of aerosol preparations, but which nevertheless should be avoided because of their unacceptable impact on the environment or other accompanying circumstances, in particular fluorocarbons and chlorofluorocarbons (CFCs).

Cosmetic preparations according to the present invention can also be present as gels, which in addition to an effective amount of the compounds used according to the present invention and solvents conventionally used therefor, preferably water, also contain organic thickeners.

Gelling agents, also called thickeners, are macromolecules which have a largely linear structure and have intermolecular forces of interaction which permit secondary and primary valence bonds between the individual molecules and thus the formation of a network-like structure. Some of them are water-soluble natural or synthetic polymers which form gels or viscous solutions in aqueous systems. They increase the viscosity of the water by either binding water molecules (hydration), or else by absorbing and encapsulating the water into their interwoven macromolecules, at the same time restricting the mobility of the water. Water-soluble polymers of this type represent a large group of chemically very different natural and synthetic polymers whose common feature is their solubility in water or aqueous media. A prerequisite for this is that these polymers have a number of hydrophilic groups that is sufficient for the solubility in water and that they are not too strongly crosslinked. The hydrophilic groups may be nonionic, anionic or cationic in nature.

Advantageous thickeners for cosmetic preparations are, for example, copolymers of C₁₀₋₃₀ alkyl acrylates and one or more monomers of acrylic acid, methacrylic acid or esters thereof. The INCI designation for compounds of this type is “acrylates/C 10-30 alkyl acrylate crosspolymer.” Particularly advantageous are the Pemulen® grades TR1, TR2 and TRZ by Goodrich (Noveon).

Carbopols are also advantageous gelling agents for preparations according to the invention. Carbopols are polymers of acrylic acid, in particular also acrylate-alkyl acrylate copolymers. Advantageous carbopols are, for example, the grades 980, 981, 984, 1342, 1382, 2984 and 5984, likewise the ETD grades 2001, 2020, 2050 and Carbopol Ultrez 10, PVM/MA decadiene crosspolymer (trade name Stabileze 06), polyglyceryl methacrylate, and polyacrylamide. Also advantageous gelling agents for such preparations are xanthan gum, polyvinylpyrrolidone, cellulose derivatives, in particular cellulose ethers, such as, for example, hydroxypropylmethylcellulose, starch and starch derivatives, hyaluronic acid, carob seed flour, silica and aluminum silicates.

The thickener usually is contained in the gel, the dispersion or the emulsion, e.g., in a concentration between about 0.01% and about 5% by weight, preferably between about 0.1% and about 2% by weight.

The acrylamidomethylpropane sulfonate polymers, AMPS polymers for short, may be commercially available as 100% active substance or as inverse thickeners.

### EXAMPLES

Cross-linked AMPS copolymers, powder form
Aristoflex AVC (ammonium acryloyl dimethyltaurate/VP copolymer, Clariant) Aristoflex HMB (ammonium acryloyl dimethyltaurate/beheneth-25 methacrylate copolymer, Clariant)
Cross-linked AMPS copolymers, inverse thickeners:
Simulgel NS (Seppic): hydroxyethyl acrylate/sodium acryloyl dimethyltaurate copolymer/squalane/polysorbate

Advantageous polymers for the present invention are described, for example, in DE 100 29 462 A1.

Acrylamidomethylpropyl sulfonic acid polymers preferred according to the present invention bear the INCI designation ammonium acryloyl dimethyltaurate/vinyl pyrrolidinon copolymers.

It may be preferred according to the invention to add complexing agents to the described preparations. Complexing agents are auxiliaries known per se in cosmetology and galenic medicine. Complexing agents, in particular chelating agents, form complexes with metal atoms; in the presence of one or more polybasic complexing agents, i.e. chelating agents, these complexes represent metallacycles. Chelating agents are compounds in which an individual ligand occupies more than one coordination site on a central atom. In this case, therefore, compounds which are normally linear are closed as a result of complex formations via a metal atom or ion to form rings. The number of bonded ligands depends on the coordination number of the central metal. A prerequisite for chelate formation is that the compound that reacts with the metal contains two or more atomic groups which act as electron donors.

The complexing agent(s) can advantageously be chosen from the group of customary compounds, preference being given to at least one substance selected from tartaric acid and anions thereof, citric acid and anions thereof, aminopolycarboxylic acids and anions thereof (such as, for example, ethylenediaminetetraacetic acid (EDTA) and anions thereof, and nitrilotriacetic acid (NTA) and anions thereof.

The complexing agent(s) is/are advantageously present in cosmetic or dermatological preparations at about 0.01% to about 10% by weight, preferably about 0.05% to about 10% by weight, particularly preferably about 0.1%-1.0% by weight, based on the total weight of the preparations.

The composition according to the invention may be present in a form which is suitable for topical application. For example, the composition may be present in the form of a cream, a lotion, a gel, an ointment, a tincture, a skin oil, a milk, a balm, a bandage impregnated with the composition, a cloth impregnated with the composition, a textile impregnated with the composition, a pad impregnated with the composition, a spray, an aerosol, a roll-on, a stick, a soft solid, a powder or a powder spray.

Advantageously the formulations according to the invention can also contain substances that absorb UV radiation in the UVB range, wherein the total quantity of the filter substances is, e.g., 0.1% by weight to 30% by weight, preferably 0.5 to 10% by weight, in particular 1 to 6% by weight, based on the total weight of the preparations.

The following examples are intended to clarify the present invention without restricting it. All quantities, proportions and percentages are based on the weight and the total quantity or on the total weight of the preparations, unless stated otherwise.

### Formula Example 1

### O/W Emulsions

| | % by | % by |
|---|---|---|
| Raw material (INCI) | weight | weight |
| PEG-40 stearate | 2.00 | 2.00 |
| Glyceryl stearate | 2.00 | 1.00 |
| Stearyl alcohol | 0.50 | 1.50 |
| Cetyl alcohol | 2.00 | 2.00 |
| C12-15 Alkyl benzoates | 2.00 | 2.00 |
| Caprylic acid/capric acid triglycerides | 1.00 | 1.00 |
| Cyclomethicone | 3.00 | 3.00 |
| Dicaprylyl carbonate | 2.00 | 2.00 |
| Lactic acid | 0.25 | 0.25 |
| Vitamin B3 (niacinamide) | 0.30 | — |
| (1R,2S,5R)-2-Isopropyl-5-methyl-N-(2-(pyridin-2- | 0.10 | |
| yl)ethylcyclohexanecarboxamide | | |
| (1R,2S,5R)-N-(4-(Cyanomethyl)-phenyl)-2-isopropyl- | | 0.2 |
| 5-methylcyclohexanecarboxamide | | |
| Genistein | | 0.10 |
| Phenoxyethanol | 0.40 | 0.40 |
| Diazolidinyl urea | 0.20 | 0.20 |
| Methyl propanediol | 4.00 | 4.00 |
| Ammonium acryloyl dimethyltaurate/VP copolymer | 0.80 | 0.80 |
| Glycerin | 5.00 | 5.00 |
| Fillers (distarch phosphate, SiO₂, talc) | 0.10 | 0.10 |
| Perfume/dyes | q.s. | q.s. |
| Water | ad 100 | ad 100 |

### Formula Example 2

### Rich Cream for Dry Skin

| | % by | % by |
|---|---|---|
| O/W night cream | weight | weight |
| Glyceryl stearate citrate | 2 | 3 |
| Shea butter | 2 | 2 |
| Stearyl alcohol | 2 | 3 |
| Cetyl alcohol | 2 | — |
| Hydrogenated coco glycerides | 2 | 2 |
| Caprylic acid/capric acid triglycerides | 2 | 2 |
| Ethylhexyl coconut fatty acid esters | 2 | 2 |
| Cyclometicone | 3 | 3 |
| Dicaprylyl ether | 2 | 2 |
| Tocopheryl acetate | 1 | 1 |
| Ubiquinone (Q10) | 0.1 | — |
| Sodium ascorbyl phosphate | 0.1 | 0.1 |
| (1R,2S,5R)-2-Isopropyl-5-methyl-N-(2-(pyridin-2- | 1 | |
| yl)ethylcyclohexanecarboxamide | | |
| (1R,2S,5R)-N-(4-(Cyanomethyl)-phenyl)-2-isopropyl- | | 0.5 |
| 5-methylcyclohexanecarboxamide | | |
| Retinyl palmitate | — | 0.1 |
| Phytosterols | | 0.2 |
| Alkyl esters of p-hydroxy benzoic acid (parabens) | 0.6 | 0.6 |
| Ethylhexylglycerin | 0.5 | 0.5 |
| Carbomer | 0.3 | 0.3 |
| EDTA | 0.2 | 0.2 |
| Glycerin | 10 | 10 |
| Water-soluble and/or oil-soluble dyes | 0.05 | 0.05 |
| Fillers/additives (SiO₂, BHT) | 0.2 | 0.2 |
| Menthoxypropanediol | 1 | 1 |
| Water | ad 100 | ad 100 |

### Formula Example 3

### W/O Emulsions

| | % by | % by |
|---|---|---|
| W/O cream for reducing itching | weight | weight |
| Polyglyceryl-3 diisostearate | 5.0 | 4.0 |
| Polyglyceryl-2 dipolyhydroxystearate | 2.5 | 3.0 |
| Cetearyl alcohol | 2 | |
| Cetyl alcohol | 2 | 3 |
| C₁₂₋₁₅ Alkyl benzoate | 8 | 5 |
| Caprylic acid/capric acid triglycerides | 6 | 6 |
| Octyldodecanol | 5 | 5 |
| Octamethyltetrasiloxane (cyclomethicone) | 2 | 6 |
| Menthoxypropanediol | 1 | 3 |
| Citric acid, sodium salt | 0.5 | 0.5 |
| Butyl methoxydibenzoylmethane | 1 | 1 |
| Ethylhexyl triazone | 1 | 1 |
| Ethylhexyl methoxycinnamate | 5 | 5 |
| (1R,2S,5R)-2-Isopropyl-5-methyl-N-(2-(pyridin-2- | 0.2 | |
| yl)ethylcyclohexanecarboxamide | | |
| (1R,2S,5R)-N-(4-(Cyanomethyl)-phenyl)-2-isopropyl- | | 0.05 |
| 5-methylcyclohexanecarboxamide | | |
| Sodium polyacrylate | 0.15 | 0.15 |
| Retinyl palmitate | 0.05 | — |
| Alkyl esters of p-hydroxy benzoic acid (parabens) | 0.1 | 0.1 |
| Glycerin | 7 | 7 |
| Additives (EDTA, BHT) | 0.6 | 0.6 |
| Perfume | q.s. | q.s. |
| Water | ad 100 | ad 100 |

### Formula Example 4

### Skin Care O/W Emulsions with Cooling Effect

| | % by | % by |
|---|---|---|
| O/W cream | weight | weight |
| Cetearyl glucoside | 2 | 3 |
| Myristyl myristate | 1 | — |
| Stearyl alcohol | 4 | 3 |
| C₁₂₋₁₅ Alkyl benzoate | 2 | 2 |
| Caprylic acid/capric acid triglycerides | 3 | 3 |
| Hydrogenated polydecene | 1 | — |
| Dicaprylyl carbonate | 3 | 3 |
| Cyclomethicone | 4 | 5 |
| Ethylhexyl methoxycinnamate | 5 | 5 |
| Ethylhexyl 2-cyano-3,3-diphenylacrylate (Octocrylene) | 3 | 3 |
| Bis-ethylhexyloxyphenol methoxyphenyl triazine | 2 | 2 |
| Menthoxypropanediol | 1.5 | 1.5 |
| Ubiquinone (Q10) | 0.05 | 0.05 |
| Urea | 3 | 3 |
| Trisodium EDTA | 0.1 | 0.1 |
| Polyvinylpyrrolidone-hexadecene copolymer | 0.4 | 0.4 |
| Alkyl esters of p-hydroxy benzoic acid (parabens) | 0.4 | 0.4 |
| (1R,2S,5R)-2-Isopropyl-5-methyl-N-(2-(pyridin-2- | 0.3 | |
| yl)ethylcyclohexanecarboxamide | | |
| (1R,2S,5R)-N-(4-(Cyanomethyl)-phenyl)-2-isopropyl- | | 0.6 |
| 5-methylcyclohexanecarboxamide | | |
| Xanthan gum | 0.1 | 0.1 |
| Carrageenan | 0.1 | 0.1 |
| Aluminum starch octenylsuccinate | 1 | 1 |
| Glycerin | 6 | 6 |
| Butylene glycol | 2 | 2 |
| Additives (talcum, BHT, dye) | 1 | 1 |
| Perfume | q.s | q.s. |
| Water | ad 100 | ad 100 |

### Formula Example 5

### Skin Care O/W Emulsions with Cooling Effect

| | % by | % by |
|---|---|---|
| Sensitive cream | weight | weight |
| Glyceryl stearate | 1 | 2 |
| Stearic acid | 3 | 3 |
| Stearyl alcohol | 2 | — |
| Cetyl alcohol | 2 | 3 |
| C₁₂₋₁₅ Alkyl benzoate | 2 | 2 |
| Caprylic acid/capric acid triglycerides | 2 | 2 |
| Macadamia oil | 1 | — |
| Myristyl myristate | 2 | — |
| Dimethicone | 2 | 0.5 |
| Hydrogenated coco glycerides | — | 1 |
| Tocopheryl acetate | 1 | 2 |
| (1R,2S,5R)-2-Isopropyl-5-methyl-N-(2-(pyridin-2- | 0.2 | |
| yl)ethylcyclohexanecarboxamide | | |
| (1R,2S,5R)-N-(4-(Cyanomethyl)-phenyl)-2-isopropyl- | | 1.0 |
| 5-methylcyclohexanecarboxamide | | |
| Fucus vesiculosus | 0.3 | 0.3 |
| Ursolic acid | 0.1 | |
| Allantoin | | 0.2 |
| Phenoxyethanol | 0.4 | 0.4 |
| Cyclodextrin | 0.3 | 0.3 |
| EDTA | 0.2 | 0.2 |
| C₁₀₋₃₀ Alkyl/acrylates crosspolymer | 0.3 | 0.3 |
| Glycerin | 5 | 5 |
| Panthenol | | 1.0 |
| Methylpropanediol | 1 | 1 |
| Additives (SiO₂, talcum, nylon-12) | 0.5 | 0.5 |
| Water | ad 100 | ad 100 |

1.-9. (canceled)

10. A method of at least one of reducing itching, reducing paraesthesias with dry skin or aged skin, and reducing dermatological paraesthesias caused by sunburn, wherein the method comprises applying to skin of a subject in need thereof a cosmetic or dermatological preparation that comprises at least one of (1R,2S,5R)-2-isopropyl-5-methyl-N-(2-(pyridin-2-yl)ethylcyclohexane-carboxamide and (1R,2S,5R)-N-(4-(cyanomethyl)-phenyl)-2-isopropyl-5-methylcyclohexanecarboxamide in an amount that is effective for at least one of reducing itching, reducing paraesthesias with dry skin and aged skin, and reducing dermatological paraesthesias caused by sunburn.

11. The method of claim 10, wherein itching is reduced.

12. The method of claim 10, wherein paraesthesias with dry skin and aged skin are reduced.

13. The method of claim 10, wherein paraesthesias caused by sunburn are reduced.

14. The method of claim 10, wherein the preparation further comprises menthoxypropanediol.

15. The method of claim 10, wherein the preparation comprises from 0.001% to 10% by weight, based on a total weight of the preparation, of at least one of (1R,2S,5R)-2-isopropyl-5-methyl-N-(2-(pyridin-2-yl)ethylcyclohexanecarboxamide and (1R,2S,5R)-N-(4-(cyanomethyl)-phenyl)-2-isopropyl-5-methylcyclohexanecarboxamide.

16. The method of claim 15, wherein the preparation comprises at least 0.01% by weight of at least one of (1R,2S,5R)-2-isopropyl-5-methyl-N-(2-(pyridin-2-yl)ethylcyclohexanecarboxamide and (1R,2S,5R)-N-(4-(cyanomethyl)-phenyl)-2-isopropyl-5-methylcyclohexanecarboxamide.

17. The method of claim 16, wherein the preparation comprises not more than 1% by weight of at least one of (1R,2S,5R)-2-isopropyl-5-methyl-N-(2-(pyridin-2-yl)ethylcyclohexanecarboxamide and (1R,2S,5R)-N-(4-(cyanomethyl)-phenyl)-2-isopropyl-5-methylcyclohexanecarboxamide.

18. The method of claim 14, wherein the preparation comprises from 0.001% to 10% by weight, based on a total weight of the preparation, of menthoxypropanediol.

19. The method of claim 18, wherein the preparation comprises at least 0.01% by weight of menthoxypropanediol.

20. The method of claim 19, wherein the preparation comprises not more than 1% by weight of menthoxypropanediol.

21. The method of claim 10, wherein the preparation further comprises at least one sunscreen agent.

22. The method of claim 10, wherein the preparation further comprises at least one antioxidant.

23. The method of claim 10, wherein the preparation further comprises at least one vitamin.

24. A method of at least one of reducing itching, reducing paraesthesias with dry skin and aged skin, and reducing dermatological paraesthesias caused by sunburn, wherein the method comprises applying to skin of a subject in need thereof a cosmetic or dermatological preparation that comprises from 0.01% to 1% by weight, based on a total weight of the preparation, of at least one of (1R,2S,5R)-2-isopropyl-5-methyl-N-(2-(pyridin-2-yl)ethylcyclohexanecarboxamide and (1R,2S,5R)-N-(4-(cyanomethyl)-phenyl)-2-isopropyl-5-methylcyclohexanecarboxamide in an amount that is effective for at least one of reducing itching, reducing paraesthesias with dry skin and aged skin, and reducing dermatological paraesthesias caused by sunburn.

25. The method of claim 24, wherein the preparation further comprises from 0.01% to 1% by weight, based on a total weight of the preparation, of menthoxypropanediol.

26. The method of claim 25, wherein the preparation further comprises at least one sunscreen agent.

27. The method of claim 25, wherein the preparation further comprises at least one antioxidant.

28. The method of claim 25, wherein the preparation further comprises at least one vitamin.

29. The method of claim 24, wherein itching is reduced.
